(19) United States
(12) Patent Application Publication
(10) Pub. No.: US 2006/0127451 A1
(43) Pub. Date: Jun. 15, 2006

(54) Coating composition

(76) Inventors: Michael Augello, Marlboro, NJ (US); Sharon Ray, Dover, NJ (US); Jian-Xin Li, New Brunswick, NJ (US); Brian Carlin, Lawrenceville, NJ (US)

Correspondence Address: Paul Fair;FMC Corporation, 1735 Market Street, Philadelphia, PA, 19103, US

(21) Appl. No.: 11/232,690

(22) Filed: Sep. 22, 2005

Related U.S. Application Data
(60) Provisional application No. 60/612,350, filed on Sep. 23, 2004.

(51) Int. Cl. A61K 9/68; A61K 9/20; A61K 31/717

(52) U.S. Cl. 424/440; 424/464; 514/57

(57) ABSTRACT

The present invention is directed to compositions containing: (i) sugar and/or a sugar substitute and (ii) microcrystalline cellulose present in an amount equal to greater than 41% of the total weight of the composition. The present invention is also directed to compositions containing: (i) sugar and/or a sugar substitute and (ii) microcrystalline cellulose having an average particle size equal to or less than 20 microns and present in an amount equal to or greater than 20% of the total weight of the composition. The compositions of the present invention are useful in coating a wide variety of solid forms such as pharmaceutical dosage forms, veterinary dosage forms, nutraceutical dosage forms, food, industrial material, cosmetic material or oral care material. The compositions of the invention are also useful as films such as cast films. The present invention is also directed to methods of making such compositions, as well as methods of coating such compositions on solid forms.

This application claims the benefit of U.S. Provisional Application No. 60/612,350, filed Sep. 23, 2004.

### FIELD OF THE INVENTION

The present invention is directed to compositions containing: (i) sugar and/or a sugar substitute and (ii) microcrystalline cellulose present in an amount equal to greater than 41% of the total weight of the composition. The present invention is also directed to compositions containing: (i) sugar and/or a sugar substitute and (ii) microcrystalline cellulose having an average particle size equal to or less than 20 microns and present in an amount equal to greater than 20% of the total weight of the composition. The compositions of the present invention are useful in coating a wide variety of solid forms such as pharmaceutical dosage forms, veterinary dosage forms, nutraceutical dosage forms, food, industrial material, cosmetic material or oral care material. The compositions of the invention are also useful as films such as cast films. The present invention is also directed to methods of making such compositions, as well as methods of coating such compositions on solid forms.

### BACKGROUND OF THE INVENTION

Sugar coatings have long been used on pharmaceutical dosage forms and on foods. In coating pharmaceutical dosage forms, such sugar coatings have often involved a complicated and lengthy process involving a number other coatings and significant drying times.

U.S. Pat. No. 5,547,948 discloses the use of microcrystalline cellulose/sugar coatings can control the release rate of hormonal steroids when the microcrystalline cellulose is used at very low levels; i.e., 0.1 to 3 and further discloses the use of microcrystalline cellulose/sugar coatings where the microcrystalline cellulose is present in an amount of 7.5 to about 15%.

JPA-38[1963]-7037 discloses a mixture of microcrystalline cellulose and sugar wherein the microcrystalline cellulose has a particle size diameter no larger than 50 microns and is present in and 0.5 to 40 wt %.

### SUMMARY OF THE INVENTION

The present invention is directed to compositions containing: (i) sugar and/or a sugar substitute and (ii) microcrystalline cellulose present in an amount equal to greater than 41% of the total weight of the composition. The present invention is also directed to compositions containing: (i) sugar and/or a sugar substitute and (ii) microcrystalline cellulose having an average particle size equal to or less than 20 microns and present in an amount equal to or greater than 20% of the total weight of the composition. The compositions of the present invention are useful in coating a wide variety of solid forms such as pharmaceutical dosage forms, veterinary dosage forms, nutraceutical dosage forms, food, industrial material, cosmetic material or oral care material. The compositions of the invention are also useful as films such as cast films. The present invention is also directed to methods of making such compositions, as well as methods of coating such compositions on solid forms.

### DETAILED DESCRIPTION OF THE INVENTION

In a first embodiment, the present invention is directed to compositions containing: (i) sugar and/or a sugar substitute and (ii) microcrystalline cellulose present in an amount equal to greater than 41% of the total weight of the composition.

The amount of microcrystalline cellulose present in the first embodiment may also include an amount greater than 45%, 50%, 55%. Typical examples of microcrystalline cellulose that may be used in the first embodiment include Avicel® PH 101 having an average particle size less equal to or less than 50 microns and Avicel® PH 105 having an average particle size less than 20 microns—all of which are on sale by FMC Corporation.

A film may be prepared from the composition of the first embodiment such as a cast film, and such films may contain a wide variety of materials such as pharmaceutical and nutraceutical actives, foods, cosmetics, industrial materials, oral care materials, etc.

In a second embodiment, the present invention is directed to compositions containing: (i) sugar and/or a sugar substitute and (ii) microcrystalline cellulose having an average particle size equal to or less than 20 microns and present in an amount equal to or greater than 20% of the total weight of the composition.

The amount of microcrystalline cellulose present in the second embodiment may also include an amount greater than 30%, 40%, 45%, 50%, 55%. Typical examples of the sugar or sugar substitute include those set forth above. A typical example of the microcrystalline cellulose that may be used in the second embodiment would be Avicel® PH 105 having an average particle size less than 20 microns.

The compositions of the first and second embodiments may consist only of the microcrystalline cellulose and sugar components, but they may also further contain water or other additives such as flavorants, colorants, plasticizers, surfactants and fillers, as well as pharmaceutical and nutraceutical actives, foods, cosmetics, industrial materials, oral care materials, etc.

The compositions of the present invention are useful in coating a wide variety of solid forms such as pharmaceutical dosage forms, veterinary dosage forms, nutraceutical dosage forms, confectionary, food, industrial material, cosmetic material or oral care material, agriculturals.

The compositions of the first and second embodiments are also useful as films such as cast films.

The compositions of the first and second embodiments may be prepared as illustrated in the examples below.

The compositions of the first and second embodiments may be coated on solid forms using conventional techniques such as spray coating. Examples of such processes are set forth below in the examples. The coatings of the inventions can be applied using a process that enables the use of spray coating in place of existing commercial techniques using non-perforated pans and significantly reduces processing time over conventionally used sugar coating processes. For example, solid forms can be coated in less than 8 hours. Typical solids for the first and second compositions when placed in suspensions are 20-75 wt % based on total weight of the suspension, more particularly, 30-50 wt %, more particularly, 40 wt %.

The present invention is also directed to solid forms comprising a coating layer thereon wherein the coating layer comprises the compositions of the first or second embodiments and, optionally, one or more coating layers (such as a seal coat) between the solid form and the coating composition of the first and second embodiment. There may further be at least one coating layer applied on top of the coating composition of the first or second embodiment (such as a top coat or smooth coat).

For example, the solid forms may be coated with only with the coating composition of the first or second embodiment. The solid form may also contain a seal coat, a sugar/microcrystalline cellulose coat and optionally a smooth coating and/or a wax coating. The seal coat can be microcrystalline cellulose/carrageenan based coatings such as those described in U.S. Pat. No. 6,432,448, U.S. Pat. No. 6,500,462, and U.S. Pat. No. 6,699,315—all of which are incorporated herein by reference. The seal coat and the sugar/MCC coat of the first and second embodiments can be clear or colored as desired.

The seal coat when used may be applied onto the core of the solid form, generally followed by the sugar/MCC coat of the first and second embodiments, though it is possible that there can be another layer or layers of coatings between the seal coat and the sugar/MCC coat of the first and second embodiments.

Optionally, a smooth coat and/or a wax coat is applied after the sugar/MCC coat of the first and second embodiments is applied, though it is possible that another layer can be between the sugar/MCC coat of the first and second embodiments and the smooth coat or the wax coat. The smooth coating and wax coating can be clear or colored. The smooth coating can be the same composition as the seal coat or different. The wax coating can be any standard polishing and/or waxing agent; e.g., carnauba wax, polyethylene glycol (polisher) and propylene glycol (polisher).

Typical commercial pharmaceutical processes involving sugar based coatings require considerable time because of various coating steps thought to be required or desired. A typical coating time involving a sugar coating could be 2-4 days depending on batch size. The present invention as mentioned above allows the manufacture of solid dosage forms having a sugar coating in significantly less time; e.g., 1.5 hours to 8 hours depending on batch size. It also provides coating compositions having desirable physical attributes and, in some cases, highly preferred elegant (smooth) coatings.

The present invention is now described in more detail by reference to the following examples, but it should be understood that the invention is not construed as being limited thereto. Unless otherwise indicated herein, all parts, percents, ratios and the like are by weight.

### EXAMPLES

### Manufacturing Procedure

Examples 1-14 below used the following experimental protocol.

The seal coating composition was prepared by first premixing the powder ingredients in a blender and then slowly adding the powder premix to deionized water under good agitation. The coating was stirred for 1 hour.

The sugar/MCC coating was prepared by slowly adding a dry premix of the microcrystalline cellulose and granulated sugar (sucrose) to deionized water to form suspension. In Examples 1-3 below, the deionized water was not heated, but the deionized water was first heated to 60° C. for more rapid sugar dissolution in Examples 4-10.

The smooth coating composition may be either a clear or colored coating composition. Colored smooth coating compositions were prepared by first preparing the clear coating then adding liquid colorant. With carrageenan-based smooth coatings, the dry powder premix was added to the deionized water, then mixed for 15 minutes prior to addition of the colorant, followed by mixing for an additional 45 minutes. The alternate sucrose color smooth coating was prepared by mixing colorant into a 70% sucrose suspension.

Coatings were applied to a 1.5 kilogram charge of ibuprofen 200 mg tablet cores in 15 inch pan using an Accela Cota “Comp-U-Coat” with #4 Baffles. The spray apparatus included #1 Binks Guns (2.0 mm Fluid Nozzle), 40100 Air Cap, having a coating delivery system of a Model Digital Console Drive #7523-50 containing Masterflex Pump #1 Pump Head, #24 Tubing, 94600. Specific coating parameters for inlet air temperature, exhaust air temperature, air flow, gun atomization pressure, pan speed, coating delivery rate and coating time are presented within the examples. In general, the pan speed and spray rate were initially low to avoid overwetting of the tablets and increased to the maximum pan speed and spray rate after approximately 0.25 weight % to 0.5 weight % of coating by total weight of the suspension was applied.

When the tablets were waxed and polished, the tablet bed was heated to approximately 100° C., the inlet and exhaust was shut off and about 1 to 2 wt % of carnauba wax by total weight of the tablet was added and the bed was allowed to roll for 1 to 2 minutes. The exhaust was turned on to remove any excess and the tablets were allowed to roll for about 5 minutes while cooling to room temperature.

Testing Procedures

Viscosity was measured with a Brookfield RVT using a #4 spindle at 20 rpm after 20 seconds. Hardness was measured using a Schleuniger tester. Disintegration was measured using USP standards. Friability was measured according to USP standards with the friability time as indicated. Dissolution was measured according to USP standards for ibuprofen—apparatus was 2 (paddle, 50 rpm, 900 ml, 0.05M phosphate buffer, pH 7.2).

### Example 1

A 10% solids seal coating composition having a viscosity of 1500 mpa-s was prepared using a powder premix of 44% Avicel® PH-105, 18% of cold-soluble iota carrageenan (visc=600 cps) and 38% of PEG 8000. A sugar build up suspension was prepared using a powder premix of 70% granulated sugar and 30% Avicel® PH-105 and cold deionized water. Tablets were coated sequentially to give seal coat (3% weight gain of the tablet) and a sugar/microcrystalline cellulose layer (30 to 40% weight gain of the tablet).

TABLE 1
Coating Parameters

| | Seal Coat | Sugar/MCC |
|---|---|---|
| Inlet Air Temperature, ° C. | 50-65 | 50-55 |
| Exhaust Air Temperature, ° C. | 31-45 | 35-41 |
| Air, CFM | 250-259 | 200-210 |
| ATM, psi | 25 | 25 |
| Pan Speed, RPM | 10-14 | 15-18 |
| Delivery Rate, gms/min | 17-72 | 16-20 |
| Coating Time, minutes | 60.0 | 40.0 |

TABLE 2
Physical Testing

| | 24-Hrs Out of Pan | Uncoated Cores |
|---|---|---|
| Tablet Weight, grams | 6.137 | 4.06 |
| Thickness, mm | 7.82 | 6.40 |
| Hardness, Kp | 10.7 | 3.2 |
| Disintegration (37° C. DI Water) | 7.30 | 0.5 |
| Friability (5.0 minutes), % | 0 | 0.143 |

TABLE 3
Dissolution Profile

| Time | 24-Hrs Out of Pan | Uncoated Cores |
|---|---|---|
| 15 minutes | 96 ± 3.0 | 97 ± 2.4 |
| 30 minutes | 99 ± 2.1 | 99 ± 1.7 |
| 45 minutes | 99 ± 1.9 | 99 ± 2.0 |
| 60 minutes | 100 ± 2.1 | 100 ± 1.6 |

### Example 2

Tablets were coated with the compositions of example 1 to give a three layer coated tablet. After the seal coating (3% weight gain) and the sugar/MCC coating (30-40% weight gain), tablets batches were coated with a smooth coat layer of 1% and 2% by weight of the total tablet weight. The clear smooth coat composition was the same as the seal coat composition of Example 1.

TABLE 4
Coating Parameters

| | Seal Coat | Sugar/MCC coat | Smooth Coat |
|---|---|---|---|
| Inlet Air | 55 | 55-60 | 55 |
| Temperature, ° C. | | | |
| Exhaust Air | 34.3-42.7 | 39.7-47.5 | 37.3-41.6 |
| Temperature, ° C. | | | |
| Air, CFM | 250-259 | 225-233 | 250-258 |
| ATM, psi | 25 | 25 | 25 |
| Pan Speed, RPM | 12 | 12 | 12 |
| Delivery Rate, | 15-25 | 10-15 | 15 |
| gms/min | | | |
| Coating Time, | 22 | 47 | 1% -- 10 |
| minutes | | | 2% -- 22 |

TABLE 5
Physical Testing

| | 1% Smooth | 2% Smooth | Uncoated |
|---|---|---|---|
| 24 Hours Out of Pan | Coat | Coat | Tablets |
| Tablet Weight, grams | 5.76 | 5.94 | 4.06 |
| Thickness, mm | 7.49 | 7.54 | 6.40 |
| Hardness, Kp | 9.9 | 10.2 | 3.2 |
| Disintegration (37° C. DI | 2.36 | 2.72 | 0.5 |
| Water) | | | |
| Friability (5.0 minutes), % | 0 | 0 | 0.143 |

### Example 3

Tablets were coated with the compositions of Example 1 to give a three layer coated tablet with varied thickness of seal coat and sugar/MCC layers. After the seal coating (5% weight gain of the tablet) and the sugar/MCC coating (20-40% weight gain of the tablet), tablet batches were coated either with a smooth coat layer of 1% or 2% by weight of the tablet weight. The clear smooth coat composition was the same as the seal coat composition of Example 1.

TABLE 6
Coating Parameters

| | Seal Coat | Sugar/MCC coat | Smooth Coat |
|---|---|---|---|
| Inlet Air | 50-55 | 50-55 | 50 |
| Temperature, ° C. | | | |
| Exhaust Air | 38-40 | 38-40 | 39-40 |
| Temperature, ° C. | | | |
| Air, CFM | 250-261 | 253-267 | 250-263 |
| ATM, psi | 25 | 25 | 25 |
| Pan Speed, RPM | 10.8 | 12-18 | 10-8 |
| Delivery Rate, | 15-25 | 15-20 | 15 |
| gms/min | | | |
| Coating Time, | 35 | 35 | 1% -- 12 |
| minutes | | | 2% -- 24 |

TABLE 7
Physical Testing

| | 1% | 2% | Uncoated |
|---|---|---|---|
| 24 Hours Out of Pan | Smooth Coat | Smooth Coat | Tablets |
| Tablet Weight, grams | 5.29 | 5.39 | 4.06 |
| Thickness, mm | 7.13 | 7.18 | 6.40 |
| Hardness, Kp | 9.2 | 9.9 | 3.2 |
| Disintegration (37° C. DI | 2.22 | 3.0 | 0.5 |
| Water) | | | |
| Friability (5.0 minutes), % | 0 | 0 | 0.143 |

TABLE 8
Dissolution Profile

| | 1% | 2% | |
|---|---|---|---|
| Time | Smooth Coat | Smooth Coat | Uncoated Cores |
| 15 minutes | 98 ± 1.6 | 96 ± 1.2 | 97 ± 2.4 |
| 30 minutes | 99 ± 1.4 | 99 ± 1.1 | 99 ± 1.7 |
| 45 minutes | 100 ± 1.5 | 100 ± 0.6 | 99 ± 2.0 |
| 60 minutes | — | — | 100 ± 1.6 |

### Example 4

Tablets were coated with the compositions of Example 1 to give a three layer coated tablet with varied thickness of seal coat and sugar/MCC layers. After the seal coating (3% weight gain) and the sugar/MCC coating (30-40% weight gain), tablet batches were coated either with a smooth coat layer of 1% or 2% by weight of the total tablet weight. The clear smooth coat composition was the same as the seal coat composition of Example 1. Dissolution performance was compared to commercial ADVIL® caplets.

TABLE 9
Coating Parameters

| | Seal Coat | Sugar/MCC coat | Smooth Coat |
|---|---|---|---|
| Inlet Air | 55-57 | 57-60 | 55 |
| Temperature, ° C. | | | |
| Exhaust Air | 37.3-42.7 | 41.3-47.7 | 38.9-41.9 |
| Temperature, ° C. | | | |
| Air, CFM | 250-256 | 219-230 | 250-253 |
| ATM, psi | 25 | 25 | 25 |
| Pan Speed, RPM | 12 | 12 | 12 |
| Delivery Rate, | 15-27 | 15-17 | 15 |
| gms/min | | | |
| Coating Time, | 21 | 45 | 1% -- 10 |
| minutes | | | 2% -- 21 |

TABLE 10
Physical Testing

| | 1% | 2% | Uncoated |
|---|---|---|---|
| 24 Hours Out of Pan | Smooth Coat | Smooth Coat | Tablets |
| Tablet Weight, grams | 5.852 | 5.79 | 4.06 |
| Thickness, mm | 7.52 | 7.50 | 6.40 |
| Hardness, Kp | 9.2 | 10.0 | 3.2 |
| Disintegration (37° C. DI | 2.35 | 2.88 | 0.5 |
| Water) | | | |
| Friability (5.0 minutes), % | 0 | 0 | 0.143 |

TABLE 11
Dissolution Profile

| | 1% | 2% | | Uncoated |
|---|---|---|---|---|
| Time | Smooth Coat | Smooth Coat | Advil ® | Tablets |
| 15 minutes | 96 ± 2.0 | 99 ± 1.2 | 97 ± 4.0 | 97 ± 2.4 |
| 30 minutes | 98 ± 1.4 | 100 ± 1.0 | 99 ± 3.6 | 99 ± 1.7 |
| 45 minutes | 99 ± 1.3 | | 100 ± 3.0 | 99 ± 2.0 |
| 60 minutes | 99 ± 1.5 | | | 100 ± 1.6 |

### Example 5

Tablets were coated with the compositions of Example 1 to give a three layer coated tablet with varied thickness of seal coat and sugar/MCC layers. After the seal coating (3% weight gain) and the sugar/MCC coating (30-40% weight gain), tablet batches were coated either with a colored smooth coat layer of 1% or 2% by weight of the total table weight. The blue smooth coat composition was prepared at 10% total solids using a 3 to 1 weight ratio of the premix solids composition of the seal coat in Example 1 to the pigment solids. The aqueous pigment dispersion used was Chroma Kote® blue with 24% pigment loading (Chris Hansen)

TABLE 12
Coating Parameters

| | | | Blue Smooth Coat |
|---|---|---|---|
| | | Sugar/MCC | (3:1 premix |
| | Seal Coat | coat | solids:pigment) |
| Inlet Air | 65-66 | 50-65 | 55-60 |
| Temperature, ° C. | | | |
| Exhaust Air | 37-43 | 42.3-46.2 | 39.3-43.5 |
| Temperature, ° C. | | | |
| Air, CFM | 248-256 | 217-238 | 247-263 |
| ATM, psi | 25 | 25 | 25 |
| Pan Speed, RPM | 12 | 12 | 12 |
| Delivery Rate, | 20-45 | 10-15 | 15-32 |
| gms/min | | | |
| Coating Time, | 14 | 60 | 1% -- 7 |
| minutes | | | 2% -- 12 |

TABLE 13
Physical Testing

| | | 1% Blue | 2% Blue | Uncoated |
|---|---|---|---|---|
| 24 Hours Out of Pan | | Smooth Coat | Smooth Coat | Tablets |
| Tablet Weight, grams | | 5.91 | 6.22 | 4.06 |
| Thickness, mm | | 7.46 | 7.61 | 6.40 |
| Hardness, Kp | | 11.4 | 10.2 | 3.2 |
| Disintegration (37° C. | | 2.44 | 3.31 | 0.5 |
| DI Water), min | | | | |
| Friability, % | (5.0 minutes) | NT | NT | 0.143 |
| | (30 minutes) | 0 | 0 | NT |

### Example 6

Tablets were coated with the compositions of Example 1 to give a three layer coated tablet with varied thickness of seal coat and sugar/MCC layers. After the seal coating (3% weight gain) and the sugar/MCC coating (30-40% weight gain) were applied, tablet batches were coated either with a colored smooth coat layer of 1% or 2% by weight of the total tablet weight. The blue smooth coat composition was prepared at 10% total solids using the 10 to 1 weight ratio of the premix composition of the clear seal coat in Example 1 to the pigment solids. The aqueous pigment dispersion used was Chroma Kote® blue with 11% pigment loading (Chris Hansen).

TABLE 14
Coating Parameters

| | | Sugar/MCC | Blue Smooth coat |
|---|---|---|---|
| | Seal Coat | coat | (10:1 premix/pigment) |
| Inlet Air | 55-60 | 60 | 60 |
| Temperature, ° C. | | | |
| Exhaust Air | 37-43 | 42-43 | 42-48 |
| Temperature, ° C. | | | |
| Air, CFM | 250-258 | 225-233 | 250-259 |
| ATM, psi | 25 | 25 | 25 |
| Pan Speed, RPM | 12 | 12 | 13 |
| Delivery Rate, | 15-30 | 15-17 | 15-25 |
| gms/min | | | |
| Coating Time, | 18 | 47 | 1% -- 12 |
| minutes | | | 2% -- 22 |

TABLE 15
Physical Testing

| | | 1% | 2% | |
|---|---|---|---|---|
| | | Smooth | Smooth | Uncoated |
| 24 Hrs Out of Pan | | coat | Coat | Tablets |
| Tablet Weight, grams | | 6.045 | 6.04 | 4.06 |
| Thickness, mm | | 7.56 | 7.54 | 6.40 |
| Hardness, Kp | | 12.9 | 12.5 | 3.2 |
| Disintegration (37° C. | | 3.11 | 4.68 | 0.5 |
| DI Water), min | | | | |
| Friability, % | (5.0 minutes) | NT | NT | 0.143 |
| | (30 minutes) | 0.06 | 0 | NT |

### Example 7

Coated tablets were prepared as in Example 5. Coated tablets were waxed and polished using 1-weight % carnauba wax.

TABLE 16
Coating Parameters

| | | | Smooth coat |
|---|---|---|---|
| | Seal Coat | Sugar/MCC | (10:1 premix/pigment) |
| Inlet Air | 55-60 | 60 | 60 |
| Temperature, ° C. | | | |
| Exhaust Air | 38-44 | 45-58 | 45-47 |
| Temperature, ° C. | | | |
| Air, CFM | 250-258 | 208-220 | 250-259 |
| ATM, psi | 25 | 25 | 25 |
| Pan Speed, RPM | 12-13 | 14 | 13 |
| Delivery Rate, | 15-30 | 15-17 | 15-25 |
| gms/min | | | |
| Coating Time, | 17 | 47 | 1% -- 10 |
| minutes | | | 2% -- 12 |

TABLE 17
Physical Testing

| | 1% | 2% |
|---|---|---|
| 24 Hours Out of Pan | Smooth Coat | Smooth Coat |
| Tablet Weight, grams | NT | 5.93 |
| Thickness, mm | NT | 7.42 |
| Hardness, Kp | NT | 10.2 |
| Disintegration (37° C. DI Water), min | NT | 3 |
| Friability, (30 minutes), % | NT | 0 |

### Example 8

A 9% solids aqueous seal coating composition was prepared using a powder premix of 75% Avicel® PH-105, 25% of cold-soluble iota carrageenan (visc=225 cps). The seal coat viscosity was 900 mPas. The sugar/MCC coating was prepared by using a powder premix of 70% granulated sugar and 30% Avicel® PH-105 by dissolving in hot (60° C.) deionized water to form a suspension. A 9% solids smooth coat composition was prepared using a 10 to 1 weight ratio of the powder premix composition used in the seal coat to pigment solids. The pigment used was Opalux® brown liquid (Colorcon). The smooth coat viscosity was 800 mPas. Tablets were coated sequentially to give seal coat (3% weight gain) and a sugar/microcrystalline cellulose layer (30 to 40% weight gain) and a smooth coat (2%). Tablets were also prepared with the same coating layers that were then waxed and polished.

TABLE 18
Coating Parameters

| | Seal Coat | Suage/MCC coat | Smooth Coat |
|---|---|---|---|
| Inlet Air | 55-60 | 60 | 60 |
| Temperature, ° C. | | | |
| Exhaust Air | 38.5-42.8 | 43.8-47.3 | 25.0-25.9 |
| Temperature, ° C. | | | |
| Air, CFM | 255-259 | 209-238 | 250-259 |
| ATM, psi | 25 | 25 | 25 |
| Pan Speed, RPM | 11 | 12 | 11 |
| Delivery Rate, | 20-40 | 8-10 | 20-30 |
| gms/min | | | |
| Coating Time, | 23 | 67 | 18 |
| minutes | | | |

TABLE 19
Physical Testing

| | 2% | Uncoated |
|---|---|---|
| 24 Hours Out of Pan | Smooth Coat | Tablets |
| Tablet Weight, grams | 4.78 | 3.07 |
| Thickness, mm | 6.27 | 6.13 |
| Hardness, Kp | 19.6 | 10.8 |
| Disintegration (37° C. DI Water), min | 3 | 0.44 |
| Friability, (30 minutes), % | 0 | 0 |

### Example 9

A 15% solids aqueous seal coating composition (spray dried) was prepared using a powder premix of 58% Avicel® PH-105, 11% of cold-soluble iota carrageenan (viscosity=275 cps) and 31% glycerin. The seal coat viscosity was 600 mPas. The sugar/MCC coating was prepared using a powder premix of 70% granulated sugar and 30% Avicel® PH-105 by dissolving in hot (60° C.) deionized water to form a suspension. The smooth composition was the same as the seal coat composition. Tablets were coated sequentially to give seal coat (3% weight gain) and a sugar/microcrystalline cellulose layer (30 to 40% weight gain) and a smooth coat (1% or 2%, respectively).

TABLE 20
Coating Parameters

| | Seal Coat | Sugar/MCC | Smooth Coat |
|---|---|---|---|
| Inlet Air | 55 | 60 | 60 |
| Temperature, ° C. | | | |
| Exhaust Air | 38.6-39.6 | 44.3-47.5 | 45.7-46.9 |
| Temperature, ° C. | | | |
| Air, CFM | 250 | 216-224 | 250-258 |
| ATM, psi | 25 | 25 | 25 |
| Pan Speed, RPM | 12 | 12 | 12 |
| Delivery Rate, | 15-20 | 14 | 15-25 |
| gms/min | | | |
| Coating Time, | 18 | 48 | 1% -- 8 |
| minutes | | | 2% -- 16 |

TABLE 21
Physical Testing

| | 1% | 2% | Uncoated |
|---|---|---|---|
| 24 Hours Out of Pan | Smooth Coat | Smooth Coat | Tablets |
| Tablet Weight, grams | 4.68 | 4.61 | 3.27 |
| Thickness, mm | 6.30 | 6.31 | 5.34 |
| Hardness, Kp | 17.6 | 18.4 | 10.8 |
| Disintegration | NT | NT | >30 |
| (37° C. DI Water), min | | | |
| Friability, % (30 minutes) | 0 | 0 | 1.87 |

### Example 10

The seal coat and sugar/MCC compositions of Example 9 were used. A smooth coat composition was prepared using a 10 to 1 weight ratio of the powder premix composition used in the seal coat to pigment solids. The pigment used was Opalux® brown liquid (Colorcon). Tablets were coated sequentially to give seal coat (3% weight gain) and a sugar/microcrystalline cellulose layer (30 to 40% weight gain) and a smooth coat (2% weight gain). Tablets were also prepared with the same coating layers then were waxed and polished.

TABLE 22
Coating Parameters

| | | | Smooth Coat |
|---|---|---|---|
| | Seal Coat | Sugar/MCC | (10:1 premix/pigment) |
| Inlet Air | 55 | 60 | 60 |
| Temperature, ° C. | | | |
| Exhaust Air | 38.6-39.6 | 44.3-47.5 | 45.7-46.9 |
| Temperature, ° C. | | | |
| Air, CFM | 250 | 216-224 | 250-258 |
| ATM, psi | 25 | 25 | 25 |
| Pan Speed, RPM | 12 | 12 | 12 |
| Delivery Rate, | 15-20 | 14 | 15-25 |
| gms/min | | | |
| Coating Time, | 18 | 48 | 1% -- 8 |
| minutes | | | 2% -- 16 |

TABLE 23
Physical Testing

| | | 2% Smooth | |
|---|---|---|---|
| | 2% Smooth | Coat with | |
| | Coat with | Pigment and | Uncoated |
| 24 Hours Out of Pan | Pigment | Wax | Tablets |
| Tablet Weight, grams | 5.46 | 5.41 | 3.27 |
| Thickness, mm | 6.68 | 6.68 | 5.34 |
| Hardness, Kp | 19.8 | 18.9 | 10.8 |
| Disintegration | NT | NT | >30 |
| (37° C. DI Water), min | | | |
| Friability, % (30 minutes) | 0 | 0 | 1.87 |

### Example 11

Tablets were coated with the composition of Example 1 to give a four-layer coated tablet with varied thickness of seal coat, MCC/sugar layer, smooth coat and pigmentation coat. After the seal coating (3% weight gain) and the MCC/sugar coating (25-40% weight gain), a smooth clear coat (2% weight gain) was added to the coating process to create a smooth surface before the pigmentation coat was added. The red pigment coating composition was prepared at 10% total solids, using a 10:1 weight ratio of the premix solids composition of the seal coat in Example 1 to the pigment solids. The aqueous pigment dispersion used was Opalux® Brown with a 13-15% pigment loading (Colorcon).

Coating Parameters

| | Seal | Sugar/ | Smooth | Pigment |
|---|---|---|---|---|
| | Coat | MCC Coat | Coat | Coat |
| Inlet Air | 70 | 86.0-86.2 | 69.5-69.9 | 69.9-70 |
| Temp, ° C. | | | | |
| Exhaust Air | 38-40 | 46.0-47.0 | 37.7-38.0 | 38-39 |
| Temp, ° C. | | | | |
| Air, CFM | 36-39 | 36-39 | 36-38 | 36-38 |
| ATM, PSI | 25.0 | 25.0 | 25.0 | 25.0 |
| Pan Speed, | 15.0 | 15.0 | 12.0 | 12.0 |
| RPM | | | | |
| Delivery | 11.25 | 11.5 | 20.0 | 11.7 |
| Rate, g/min | | | | |
| Coating | 40.0 | 65.0 | 20.0 | 35.0 |
| Time, min | | | | |

24 hours Out of Pan - Physical Testing

| | |
|---|---|
| Tablet Weight, gms | 4.59 |
| Thickness, mm | 6.285 |
| Hardness, kp | 12.23 |
| Disintegration, min | 6-9 |
| (DI Water) | |
| Friability, % | 30 minutes - 0% |

Dissolution Profile

| | |
|---|---|
| 15 | 99 ± 0.8 |
| 30 | 99 ± 0.8 |
| 45 | 100 ± 0.8 |
| 60 | — |

### Example 12

Tablets were coated with the composition of Example 1 to give a four-layer coated tablet with varied thickness of seal coat and sugar/MCC layers. After the seal coating (2% weight gain) and the sugar/MCC coating (25-35% weight gain), tablets were then coated with a clear smooth coat layer of 2% by weight of the total tablet weight. The clear smooth coat composition was the same as the seal coat composition of Example 1. The fourth layer of coating was applied using a pigmented dispersion (Opalux® Brown liquid) with a 5.0% pigment loading (Colorcon).

Coating Parameters

| | Seal | Sugar/ | Smooth | Pigment |
|---|---|---|---|---|
| | Coat | MCC Coat | Coat | Coat |
| Inlet Air | 55-57 | 55-57.3 | 55 | 55-67 |
| Temp, ° C. | | | | |
| Exhaust Air | 38-40 | 38.40-46.3 | 40-42 | 38-43 |
| Temp, ° C. | | | | |
| Air, CFM | 225 | 219-226 | 250-251 | 250-256 |
| ATM, PSI | 25.0 | 25.0 | 25.0 | 25.0 |
| Pan Speed, | 12.0 | 12.0 | 12.0 | 11-13 |
| RPM | | | | |
| Delivery | 11-17 | 8.3-21.7 | 17-20 | 25-45 |
| Rate, g/min | | | | |
| Coating | 22.0 | 59.0 | 27.0 | 29.0 |
| Time, min | | | | |

### Example 13

A 10% solids seal coating composition, having a viscosity of 1500 mPA·s, was prepared by using a powder premix of 55% Avicel® PH-105, 18% of iota carrageenan (viscosity≈600 cps) and 38% PEG 8000. A sugar build-up suspension was prepared using a powder premix of 55% Avicel® PH-105, 45% granulated sugar, and hot deionized water (80° C.), preparing a 40% solids suspension. Tablets were coated sequentially to give a seal coat (3% weight gain of the tablet), sugar/MCC layer (25-35% weight gain of the tablets), a clear smooth coat (same composition as the seal coat, 2% weight gain of the tablets), and a pigmented coat (3% weight gain of the tablets). A 10% solids smooth coat composition was prepared using a 10:1 weight ratio of the powder mix composition used in the seal coat to pigment solids. The pigment used was Opalux® Brown liquid (Colorcon).

Coating Parameters

| | Seal | Sugar/ | Smooth | Pigment |
|---|---|---|---|---|
| | Coat | MCC Coat | Coat | Coat |
| Inlet Air | 59.4-60.1 | 55.1-56.7 | 54.4-54.8 | 55.0-55.3 |
| Temp, ° C. | | | | |
| Exhaust Air | 37.6-41.8 | 40.4-43.9 | 40.2-40.4 | 40.2-41.4 |
| Temp, ° C. | | | | |
| Air, CFM | 250-264 | 221-225 | 256.0 | 247-256 |
| ATM, PSI | 25.0 | 25.0 | 25.0 | 25.0 |
| Pan Speed, | 12-22 | 15.5-25.6 | 17.0 | 21-35 |
| RPM | | | | |
| Delivery | 12.0 | 12.0-12.5 | 12.5-14 | 12.5-14 |
| Rate, g/min | | | | |
| Coating | 15.0 | 61.0 | 25.0 | 19.0 |
| Time, min | | | | |

24 hours Out of Pan - Physical Testing

| | |
|---|---|
| Tablet Weight, gms | 5.62 |
| Thickness, mm | 6.18 |
| Hardness, kp | 7.8 |
| Disintegration, min | 3.75 |
| (37° C. DI Water) | |
| Friability, % | 5 minutes - 0% |
| | 30 minutes - 0% |

Dissolution Profile

| Time | 24 Hours Out of Pan | Uncoated Cores |
|---|---|---|
| 15 minutes | 97 ± 1.6 | 97 ± 2.4 |
| 30 minutes | 99 ± 0.8 | 99 ± 1.7 |
| 45 minutes | 101 ± 1.3 | 99 ± 2.0 |
| 60 minutes | — | 100 ± 1.6 |

### Example 14

Cast films utilizing a LustreClear™ composition (e.g., MCC (44%), sodium iota carrageenan (18%), PEG (38%)) and condition under the appropriate temperature/humidity. When the films are formed, place one (1) film in an appropriate vessel. Prepare the Avicel®/sucrose suspensions of the invention in accordance with the Manufacturing Procedure herein. If desired, add to the above suspension and/or LustreClear™ film medications such as antihistamines, non-steroidal anti-inflammatories, cardiovasculars, antihypertensives, etc. Pour the Avicel®/sucrose suspensions of the invention over the cast film and, to that apply an additional LustreClear™ cast film. Place the sample in the appropriate temperature/humidity chamber and let condition. When conditioning is complete, cut the film into strips exemplifying a Fast Disintegrating Medicated Strip.

While the invention has been described in detail and with reference to specific embodiments thereof, it will be apparent to one skilled in the art that various changes and modifications can be made therein without departing from the spirit and scope thereof.

What is claimed is:

1. A composition comprising: (i) at least one sugar or sugar substitute and (ii) microcrystalline cellulose, wherein said microcrystalline cellulose is present in an amount equal to greater than 41% of the total weight of the composition.

2. A composition as in claim 1; wherein said microcrystalline cellulose is present in an amount of greater than 45% of the total weight of the composition.

3. A composition as in claim 1, wherein said microcrystalline cellulose is present in an amount of greater than 50% of the total weight of the composition.

4. A composition as in claim 1, wherein said microcrystalline cellulose is present in an amount of greater than 55% of the total weight of the composition.

5. A composition as in claim 1, wherein said microcrystalline cellulose has an average particle size equal to or less than 50 microns.

6. A composition as in claim 1, wherein said microcrystalline cellulose has an average particle size equal to or less than 20 microns.

7. A solid form comprising a coating layer thereon wherein said coating layer comprises the composition of claim 1 and, optionally, one or more coating layers between the solid form and the coating composition of claim 1.

8. A solid form of claim 7 wherein said solid form is a pharmaceutical dosage form, veterinary dosage form, nutraceutical dosage form, confectionary, food, industrial material, cosmetic material or oral care material.

9. A film comprising the composition of claim 1.

10. A film as in claim 9, wherein said film is a cast film.

11. A method for coating a solid form optionally containing one or more coatings, comprising applying the composition of claim 1 directly onto the solid form or optionally onto the solid form containing one or more coatings.

12. A composition comprising: (i) at least one sugar or sugar substitute and (ii) microcrystalline cellulose, wherein said microcrystalline cellulose has an average particle size equal to or less than 20 microns and is present in an amount equal to or greater than 20% of the total weight of the composition.

13. A composition of claim 12, wherein said microcrystalline cellulose is present in an amount of equal to or greater than 30% of the total weight of the composition.

14. A composition of claim 12, wherein said microcrystalline cellulose is present in an amount of equal to or greater than 40% of the total weight of the composition.

15. A solid form comprising a coating layer thereon wherein said coating layer comprises the composition of claim 12, and, optionally, one or more coating layers between the solid form and the coating composition of claim 12.

16. A solid form of claim 15 wherein said solid form is a pharmaceutical dosage form, veterinary dosage form, nutraceutical dosage form, confectionary, food, industrial material, cosmetic material or oral care material.

17. A film comprising the composition of claim 12.

18. A film as in claim 17, wherein said film is a cast film.

19. A method for coating a solid form optionally containing one or more coatings, comprising applying the composition of claim 12 directly onto the solid form or optionally onto the solid form containing one or more coatings.
